(19) United States
(12) Patent Application Publication
(10) Pub. No.: US 2014/0114341 A1
(43) Pub. Date: Apr. 24, 2014

(54) EXPANDABLE DILATOR

(71) Applicants: Kyle Wolff, Cottage Grove, MN (US); Daniel McPhillips, Ham Lake, MN (US); Garrett Ganske, Lino Lakes, MN (US); Phil Olsen, Plymouth, MN (US)

(72) Inventors: Kyle Wolff, Cottage Grove, MN (US); Daniel McPhillips, Ham Lake, MN (US); Garrett Ganske, Lino Lakes, MN (US); Phil Olsen, Plymouth, MN (US)

(21) Appl. No.: 14/061,714

(22) Filed: Oct. 23, 2013

Related U.S. Application Data
(60) Provisional application No. 61/717,600, filed on Oct. 23, 2012.

(51) Int. Cl. A61M 29/00

(52) U.S. Cl. CPC A61M 29/00
USPC 606/198

(57) ABSTRACT

An expanding dilator according to certain embodiments includes an expanding portion at its distal end. The expanding portion may expand once placed in the disc space thus seating the dilator in position. The expanding dilator may also include a stop feature to prevent the dilator from being placed too far. The expanding portion may expand by actuation of a knob or other actuator on the proximal end of the dilator. The expanding portion may expand incrementally.

### PRIORITY

This application claims the benefit of U.S. Provisional Application Ser. No. 61/717,600, filed on Oct. 23, 2012, which is hereby incorporated herein by reference in its entirety.

### FIELD

The present invention generally relates to dilators. More particularly, the present invention relates to an expandable dilator useful for creating an access path to an intervertebral space.

### BACKGROUND

A variety of tools are available for surgeons for dilating tissue to create a surgical access are known. However there is a continuing need to provide improved tools for the same, especially for creating a percutaneous access path to an intervertebral space.

### SUMMARY

An expanding dilator according to certain embodiments includes an expanding portion at its distal end. The expanding portion may expand once placed in the disc space thus seating the dilator in position. The expanding dilator may also include a stop feature to prevent the dilator from being placed too far. The expanding portion may expand by actuation of a knob or other actuator on the proximal end of the dilator. The expanding portion may expand incrementally.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 depicts a view of an embodiment of the present invention.

FIG. 2 depicts a view of an embodiment of the present invention.

FIG. 3 depicts a view of an embodiment of the present invention.

FIG. 4 depicts a view of an embodiment of the present invention.

FIG. 5 depicts a view of an embodiment of the present invention.

FIG. 6 depicts a view of an embodiment of the present invention.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims.

### DETAILED DESCRIPTION

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention. It should be appreciated that, in the following embodiments and the attached drawings, elements unrelated to the present invention are omitted from depiction; and dimensional relationships among individual elements in the attached drawings are illustrated only for ease of understanding, but not to limit the actual scale.

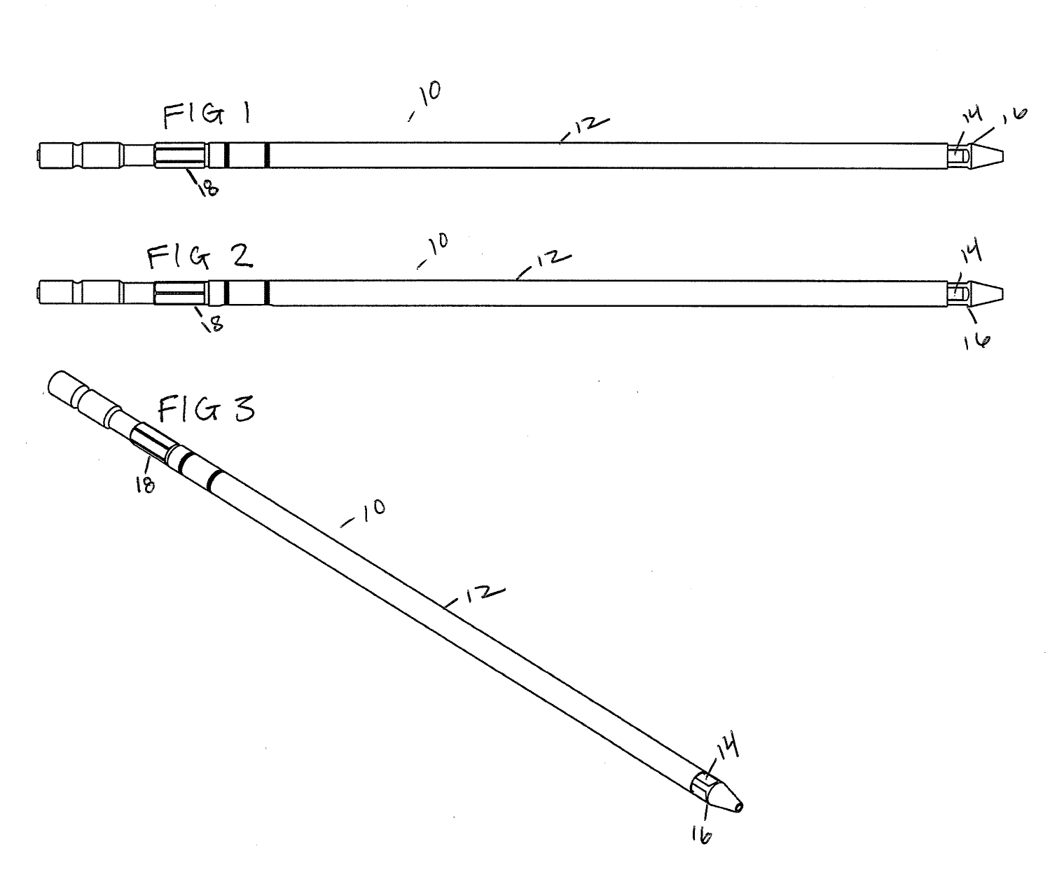

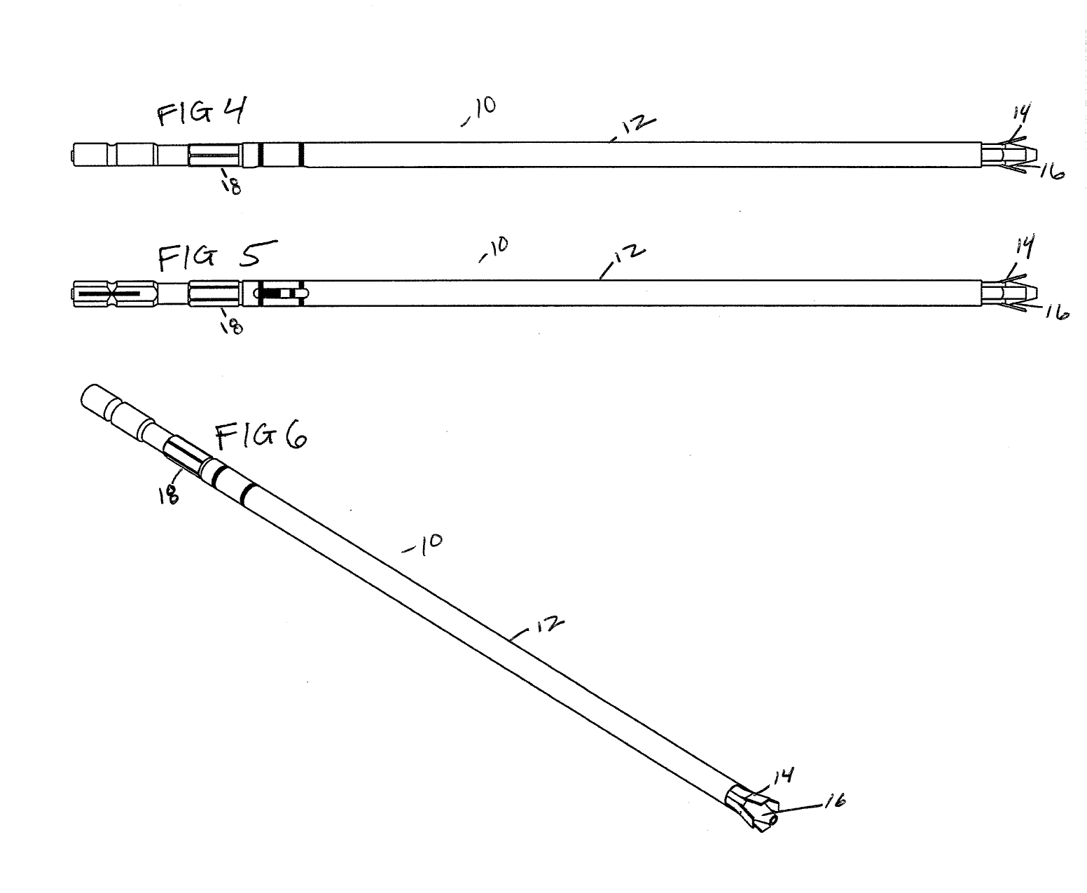

The present invention is directed to an expanding dilator for dilating tissue to create a surgical access. Expanding dilator 10 of the present invention as shown in FIGS. 1-6 includes a body portion 12 and an expanding portion 14 at a distal end 16. In an embodiment of the present invention, dilator 10 may be used to create a percutaneous access path to an intervertebral space. According to an embodiment of the present invention, expanding portion 14 may include a plurality of blades. Expanding portion 14 may expand once placed in the disc space, thus seating dilator 10 in position. The expanding distal tip portion 14 may engage tissue within the interbody disc space to prevent movement of dilator 10 once engaged with the tissue. The expanding dilator 10 of the present invention may also include a stop feature to prevent the dilator from being placed too far. The expanding portion may expand by actuation of a knob 18 or other actuator on the proximal end of the dilator. The expanding portion may expand incrementally from a fully closed position to a fully open position and any position there between.

In use, expanding dilator 10 may be used to create and maintain a percutaneous access path to an intervertebral disc space while mitigating the risk of neural impingement. According to one embodiment of the invention, dilator 10 may be inserted through a percutaneous skin incision from an oblique approach, although other approaches are contemplated. Dilator 10 may then be advanced into the center of the disc space. Expanding portion 14 may then be actuated by turning actuator knob 20, or other actuation mechanism, until expanding portion 14 has firmly engaged into the disc tissue. Sequential dilators may then be placed sequentially over expanding dilator 10 until the desired dilation for surgical access is reached. A working channel or portal, such as is disclosed in co-pending patent application Ser. No. 13/544,715, the entirety of which is incorporated by reference herein, may then be placed over the expanding dilator. Once the working portal is placed, expanding portion 14 may be retracted and expanding dilator 10 may be removed, allowing the surgeon to perform decompression, discectomy and interbody fusion device placement through the working channel as desired.

U.S. patent application Ser. No. 12/650,889, filed Dec. 31, 2009, entitled SYSTEM AND METHOD FOR PERFORMING PERCUTANEOUS SPINAL INTERBODY FUSION (U.S. Pub. App. No. US 2010/0318134 A1) is hereby incorporated by reference in its entirety as part of this application.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

What is claimed is:

1. An expandable dilator for use in creating an access to an intervertebral disc comprising:
a dilator body having a proximal end and a distal end;
the distal end including an expandable portion disposed thereon;
the expandable portion configured to engage tissue of an intervertebral disc; and
the proximal end including an actuator operably connected to the expandable portion such that the actuator moves the expandable portion from a closed position to an open position.

2. The dilator of claim 1 wherein the expandable portion comprises a plurality of blades.

3. The dilator of claim 1 wherein the expandable portion is incrementally expandable from a closed position to an open position.

4. A method of creating an access into an intervertebral disc comprising:
inserting an expandable dilator having an expanding portion, through a percutaneous skin incision;
advancing the dilator into the disc space; and
actuating the expanding portion to firmly engage the dilator into the disc space.

5. The method of claim 4, further including the step of placing sequential dilators sequentially over the expanding dilators to reach the desired dilation.

6. The method of claim 4, further comprising the step of placing a working portal over the expanding dilator.

7. The method of claim 6, further including performing a decompression.

8. The method of claim 6, further including performing a discectomy.

9. The method of claim 6, further including placing an interbody device.

10. The method of claim 4, further comprising closing the expanding dilator and removing the expanding dilator.

11. The method of claim 4, further including accessing the disc from an oblique approach.

12. The method of claim 4, further including accessing the disc from a lateral approach.
